(19) United States
(12) Patent Application Publication
(10) Pub. No.: US 2021/0379582 A1
(43) Pub. Date: Dec. 9, 2021

(54) REAL TIME DEVICE AND HYBRID METHOD FOR DETECTING AND IDENTIFYING HUMAN CORONAVIRUS IN SAMPLE SPECIMENS

(71) Applicant: Pelican Diagnostics Inc., Burlington (CA)

(72) Inventor: Raj Reddy, Burlington (CA)

(21) Appl. No.: 16/894,746

(22) Filed: Jun. 5, 2020

(51) Int. Cl. B01L 3/00; G01N 33/543

(52) U.S. Cl. CPC B01L 3/5029; B01L 3/5457; G01N 33/54386; B01L 2200/0621; B01L 2200/0689; B01L 2300/0861; B01L 2200/16; B01L 2300/025; B01L 2300/0663; B01L 2300/0645; B01L 2200/141

(57) ABSTRACT

This disclosure relates to portable devices for detecting various virus antibodies for the detection of virus diseases. Viral infections, such as those from SARS-CoV family viruses, HIV family viruses, and other family viruses can be detected by their antibodies or DNA in a clinical specimen.

### TECHNICAL FIELD

This disclosure relates to various virus antibodies and antigens for the detection of viral diseases using a portable device. Diagnosis of viral infections such as those based on SARS family viruses (e.g., SARS-COV2), HIV family viruses or other family viruses can be detected sequentially by their antibodies, antigens or RNA in a clinical specimen, such as saliva or blood. Developing an electrochemical multi-array biosensor for virus detection offers a possibility to create a low-cost and ultrasensitive and selective sensor as a point of care method to detect and quantify the antibody compounds in a sample specimen in real-time.

### BACKGROUND OF THE INVENTION

Several assays for virus detection are currently in use, including, but not limited to immunofluorescence assays, protein microarray assays, reverse transcription loop mediated isothermal amplification assays (RT-LAMP), viral plaque assays, Hem agglutination assays, viral flow cytometry (FCM) and enzyme linked immunosorbent assays (ELISA). Despite the high sensitivity of these methods, they are not suitable for large scale screening for multiple samples because of their high cost and long analysis time. Moreover, these methods require skilled personal to perform the assays and are not suitable for point-of-care testing.

Nanobiotechnology plays a potential role in clinical applications, particularly in the development of biosensors for the detection of pathogenic microorganisms. Various immunosensors have been reported for the detection of viruses using different transducers as improved alternatives to traditional assays.

For instance, the detection of SARS associated coronavirus (SARS-CoV) in sputum in the gas phase was done by piezoelectric immunosensor. This work was based on the binding of horse polyclonal antibody of SARS-CoV to a piezoelectric crystal surface through protein A. The mass of the crystal would change as the virus was bound and the shift in the frequency was recorded. A localized surface plasmon coupled fluorescence (LSPCF) fiber-optic biosensor was also developed for the detection of SARS corona virus (SARS-CoV) nucleocapsid protein N. LSPCF was used with sandwich immunoassay technique. A label-free RNA amplification and detection method was developed for the detection of MERS-CoV by using a bio-optical sensor. The level of detection (LOD) of this assay was 10 times more sensitive than the RT-PCR method. Another new generation system was developed for the robust and facile diagnosis of MERS-CoV based on an isothermal rolling circle amplification (RCA) method. However, these methods are still time consuming and costly which limits their wide applications.

Electrochemical immunosensors have become an appealing choice due to their high sensitivity, low-cost, ease of use and possibility of miniaturization. Different electrochemical immunosensors for influenza virus were reported using differential pulse voltammetry (DPV), electrochemical impedance spectroscopy (EIS), linear sweep voltammetry, chronoamperometry and cyclic voltammetry

An electrochemical immunosensor based on reduced graphene oxide (RGO) that was integrated with a microfluidic chip for label-free detection of an influenza virus (e.g., H1N1) was also reported as showing good selectivity and enhanced detection limits. Human immunodeficiency virus (HIV) was detected using DPV on glassy carbon electrodes (GCE) modified with multi-walled carbon nanotubes (MWCNTs). Human papillomavirus (HPV) was also detected using glassy carbon electrodes modified with graphene/Au nanorod/polythionine via DPV and EIS.

Gold nanoparticles (AuNPs) are the most stable metal nanoparticles, due to their unique optical, electronic, and catalytic activity, as well as their high biocompatibility properties and enhanced electron transfer rate. Therefore, they have been shown to have wide spread application in various electrochemical biosensors. Gold nanoparticles can be prepared by the chemical or electrochemical reduction of gold salt. Electrode deposition of AuNPs on the surface of carbon electrodes is appealing due to being direct, fast and easy.

An important application of antibody compound detection in a sample of subjects is compliance with same family virus diagnostics. Frequently, patients, due to memory loss or simple forgetfulness, fail to ingest prescribed medications in a timely fashion, or at all, which can lead to serious medical issues. Furthermore, health care professionals, who treat such patients, are not aware of the lack of compliance, which may prevent proper remedial action. Currently, substances can be detected in the breath of a patient by a number of methods, but none of these methods measure patient compliance directly in real time. Pre-existing methods require burdensome sample collection and subsequent analysis with delays in reporting of results.

Accordingly, there exists a need for automated portable devices and methods for directly detecting antigen compounds in the saliva of virus infected patient, which provide analytical results in real time with concurrent reporting to remote users, such as, for example, health care professionals. Simple miniaturized devices integrated with biosensors provide significant benefits and may be commercialized as a handheld device for clinical use in a point of care setting. Such devices and methods would also be of significant value in measuring patient compliance with pharmaceutical regimens and/or determining active infection. The same sensor system could also be used for quantitative and qualitative analysis of blood or other bodily fluid samples for antibodies, e.g., for a virus.

### BRIEF DESCRIPTION OF THE INVENTION

Disclosed herein are portable devices and methods for diagnosing SARS CoV family virus, HIV family virus or other family viruses by detecting and identifying their antibody compounds in saliva or blood. Also disclosed herein are biomolecular immobilized sensor substrates with novel sensing elements.

In one aspect, a portable device for detecting and identifying antibody compounds along with providing diagnosis of same family virus in a saliva or blood sample is provided. The device includes sample wells, a disposable biosensor connected to an electronic board module, which collects data that detects and identifies the compound in the saliva or blood (sample specimen) of the subject, a communication apparatus connected to the sensor module, which can transmit the data collected by the biosensor to an external processing apparatus, and a battery disposed in the housing connected to the sensor module and the communication apparatus. In some embodiments, a processing apparatus is electrically or wirelessly connected to the communication apparatus. In some embodiments, the processing apparatus analyzes data transmitted by the communication apparatus to detect and identify the one or more antibody compounds in the saliva or blood of the subject.

In some embodiments, the one or more antibody compounds are selected from the group consisting of anti-gp41, anti-gp120, SC2A, IgG, anti-M2, BCN antibodies, monoclonal antibodies (MAbs), anti-CD4bs, ADCVI antibody, anti-HA, anti-FLAG, SARS-CoV-2-N antibody, SARS-CoV-2 nucleocapsid antibody, SARS-CoV-2-S antibody, SARS-CoV-2 N Ab (IgG), spike glycoprotein antibody, ACE 2 antibody, gp150 antibody, CD147 antigen antibody, serine 2 antibody, MERS-CoV spike (S) protein, anti-HA antibody, anti-rhinovirus antibody, anti-HCV antibodies and HCoV-229E antigen protein. In some embodiments, the one or more antibody compounds are SARS-CoV-2 antibodies, e.g., SARS-CoV-2-nucleocapsid antibody, SARS-CoV-2-spike antibody, SARS-CoV-2 membrane antibody, and SARS-CoV-2 envelope antibody.

In another aspect, a method for detecting and identifying antibody compounds of same family virus in the sample specimen of a subject is provided. The method includes transmitting a sample specimen (e.g., blood or saliva) of a subject collected in sample wells to a housing, where the housing includes a sensor module, a communication apparatus and a battery. The method further includes collecting data about the identity of the compound with the sensor module, transmitting the data with the communication apparatus to a processing apparatus and analyzing the communicated data with the processing apparatus to detect and identify the one or more compounds in the sample specimen of the subject.

In still another aspect, an immobilization for a sensor substrate is provided. The immobilization includes a nanoparticle, one or more marking compounds embedded in the carbon-based nanomaterials, and a polymer matrix.

In still another aspect, a sensor, e.g., a biosensor, is provided. The sensor includes one or more antibodies on a substrate form and immobilized which includes a functionalized inorganic metallic oxide nanoparticle and a polymer matrix.

In still another aspect, a sensor is provided. The sensor includes one or more of a counter electrode, a working electrode which includes multi-walled carbon nanotubes that are attached to one or more biological molecules, and a reference electrode. The sensor further includes a support on which the electrodes are disposed.

In still another aspect, a sensor is provided. The sensor includes an electrochemical three electrode system, which includes multi-walled carbon nanotubes that are attached to one or more antibody molecules which the electrodes are disposed.

In still another aspect, a sensor is provided. The sensor includes an electrochemical three electrode system. The three electrode system may include magnetic nanoparticles and antibody functionalized carbon base-gold nanoparticle hybrid electrodes which are disposed.

In still another aspect, a sensor is provided. The sensor includes an electrochemical three electrode system which includes two different shape antibody functionalized gold nanoparticles which the electrodes are disposed.

Other embodiments, systems, methods, devices, aspects, and features of the disclosure will become apparent to those skilled in the art from the following detailed description, the accompanying drawings, and the appended claims.

### BRIEF DESCRIPTION OF THE FIGURES

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

The patent or application file contains at least one drawing executed in color. Copies of this patent or patent application publication with color drawings will be provided by the Office upon request and payment of the necessary fee.

FIG. 1 illustrates an example of a portable device which identifies and detects antibody compounds in a sample specimen of a subject.

FIG. 2 illustrates a process of identifying and detecting one or more compounds in a sample specimen of a subject.

FIG. 3 illustrates a cross sectional view of a portable device which identifies and detects one or more compounds in the breath of a subject.

FIG. 4 illustrates an example of a sensor which may be used in the sensor module of a portable device which identifies and detects one or more compounds in the sample specimen of a subject.

FIG. 5 illustrates a sensor which includes an immobilized biomolecule, which may be used in the sensor module of a portable device which identifies and detects one or more antibody compounds in the sample specimen of a subject.

FIG. 6 shows DPV responses for target antibody concentrations ranging from 50 copies per ml to 680×10⁶ copies per ml which is one of the working electrodes.

The following detailed description includes references to the accompanying drawings, which form part of the detailed description. The drawings depict illustrations, in accordance with example embodiments. These example embodiments, which are also referred to herein as “examples,” are described in enough detail to enable those skilled in the art to practice the present subject matter. The example embodiments may be combined, other embodiments may be utilized, or structural, logical, and electrical changes may be made, without departing from the scope of the claimed subject matter. The following detailed description is, therefore, not to be taken in a limiting sense, and the scope is defined by the appended claims and their equivalents.

### DETAILED DESCRIPTION OF THE INVENTION

Illustrated embodiments herein are directed to systems, methods, and devices for detecting and identifying certain substances, such as antibody compounds in the sample specimen of a subject or person in real-time. Further, certain embodiments of the disclosure can be directed to systems, methods, and devices for virus diagnosis of a subject or person. Technical effects of certain embodiments of the disclosure may include providing diagnosis and treatment for particular health conditions related to the detection and identification of certain substances, such as an antibody or DNA in the sample specimen of a subject or person in real-time.

Novel sensor technologies, such as nanocompositions with sensing elements, can be combined with mobile communication devices, such as smart phones, and cloud computing to create technical solutions for respiratory analysis, diagnosis, and subsequent treatment. Novel sensors used in combination with the processor of a smart phone and/or remote server, and a biomarker processing module or engine with a neural network or pattern matching algorithm, can be used to detect antibodies or virus DNA from sample specimens. Embodiments of the disclosure can have many useful and valuable applications in the biomedical industries, health care and medical care sectors.

Health conditions that can be detected by certain embodiments of the disclosure can include, for example, but are not limited to, adenovirus, enterovirus, human coronavirus, human metapneumovirus, rhinovirus (RV), influenza, parainfluenza and respiratory syncytial virus (RSV), severe acute respiratory syndrome coronavirus (SARS-CoV), middle East respiratory syndrome coronavirus (MERS-CoV), coronavirus, severe acute respiratory syndrome coronavirus 2 (SARS-CoV-2), adeno-associated virus, aichi virus, banna virus, bunyamwera virus, bunyavirus, cercopithecine herpesvirus, chandipura virus, dengue virus, dhori virus, dugbe virus, duvenhage virus, eastern equine encephalitis virus, ebolavirus, echovirus, encephalomyocarditis virus, GB virus C/hepatitis G virus, hantaan virus, hepatitis, human immunodeficiency virus, lassa virus, lymphocytic choriomeningitis virus, mayaro virus, measles virus, mengo encephalomyocarditis virus, mokola virus, nipah virus, rabies virus, rotavirus, rotavirus, rubella virus, salivirus, sapporo virus, seoul virus, variola virus, yellow fever virus, zika virus, etc.

As used herein, “target biomolecule” refers to signal and signal patterns associated with concentrations or amounts of certain substances associated with diagnosing or treating a health condition.

As used herein, “real-time” refers to an event or a sequence of acts, such as those executed by a computer processor that are perceivable by a subject, person, user, or observer at substantially the same time that the event is occurring or that the acts are being performed. By way of example, if a neural network receives an input based on sensing and identifying a target antibody, a result can be generated at substantially the same time that the target molecule was sensed and identified. The real-time processing of the input by the neural network may have a slight time delay associated with converting the sensed compound to an electrical signal for an input to the neural network; however, any such delay may typically be less than 1 minute and usually no more than a few seconds.

One skilled in the art will recognize that various embodiments of the disclosure discuss the analysis of antibody compounds, though certain embodiments of the disclosure can also be used for the analysis of viruses.

Disclosed herein are portable devices and methods for detecting and identifying antibody compounds in one or more sample specimens of a subject. Also disclosed herein are immobilization methods with different antibody for biosensor substrates.

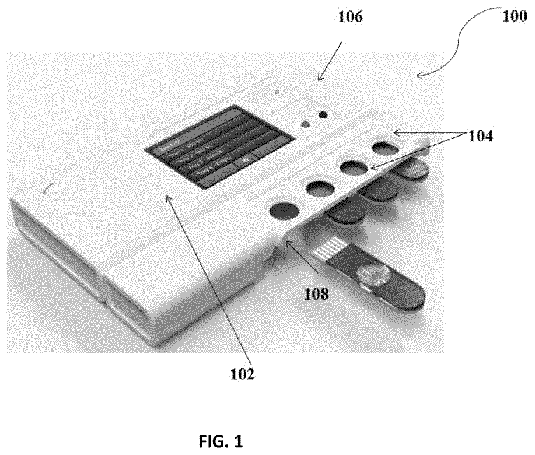

Referring to FIG. 1, illustrated is an example of a portable device 100, which may be used to detect and identify one or more compounds, e.g., one or more antibody compounds, in a sample specimen of a subject. The example device depicted in FIG. 1 contains housing 102 which includes a sample well 104, a sensor module (not shown), a communication apparatus (not shown), a charger 106 and a sensor tray 108. As will be apparent to those of skill in the art, other designs and configurations of such a portable device are possible and the above illustration is in no way limiting.

In some embodiments, a portable device, such as the one illustrated in FIG. 1, collects data about the presence and identity of antibodies of a virus in a sample specimen of a subject with a sensor module. In some aspects, the sensor module converts collected data to a signal (e.g., provides significantly high electrical conductivity, thermal conductivity, optical conductivity, etc.), and transmits the data via a communication apparatus to a processing apparatus which analyzes the data to provide information about the presence and identity of antibody compounds in the sample specimen of the subject. The processing apparatus may receive signals providing information about the presence and identity of one or more antibodies in a sample specimen of a subject and, in some aspects, may transmit the presence and identity of the one or more compounds to a display.

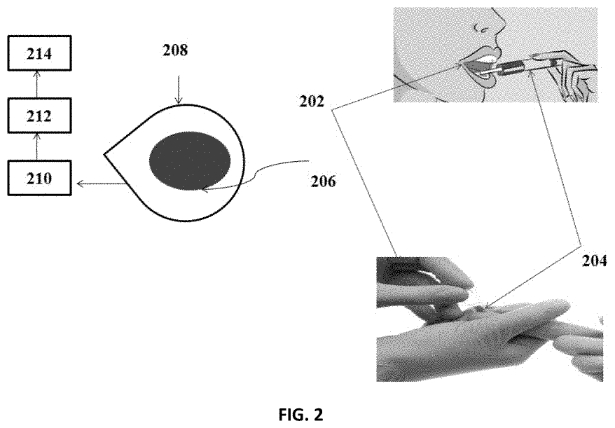

The process described herein is illustrated in FIG. 2. In one embodiment, the subject 202 introduces or provides, for example, a sample specimen 204 including one or more antibodies compounds, into housing 208 via sample well 206. In some aspects, the sample specimen is breath, saliva, blood, urine, or other bodily fluid. In some aspects, the breath of the subject enters the housing 208, which includes a battery (not shown), a sensor module (not shown), and a communication apparatus (not shown), which transmits the data (e.g., by wireless or electrical means) collected by the sensor module, for example, to the cloud 210. In one embodiment, the data may be stored in the cloud 210 and may be accessed by processing apparatus 212, which detects and identifies the compound in the breath of the subject and communicates the above result via a display to a user. In some embodiments, the communication apparatus directly transmits data to the processing apparatus 212, thus bypassing cloud 210. In certain aspects, the process illustrated in FIG. 2 occurs in real time (i.e., at substantially the same time as the event is occurring, with any delay being minimal, for example less than one minute). However, in some aspects, data may be stored in the cloud before being processed by the processing unit and displayed to a user.

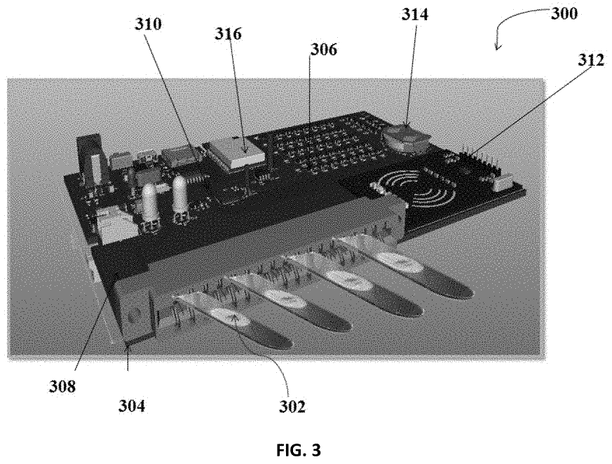

A portable device 300 is illustrated in a sectional view in FIG. 3. The device 300 has a sample well 302 through which a subject provides a sample specimen, which is connected to housing 304. Generally, housing 304 may be constructed from any material compatible with the processes described herein and may be substantially solid or hollow. In some embodiments, housing 304 is fabricated from plastic and includes a casing which is constructed of molded or printed plastic and serves to optimally position the various sensors disposed in housing 304. Housing 304 includes sensor module 306, which in one embodiment, includes a sensor array and is attached to the casing. In some embodiments, sensor module 306 is made of screen printed electrodes including six working electrodes, counter electrodes and reference electrodes. In some aspects, the electrodes are connected to a main circuit 308 with printed circuit board (PCB) 310. PCB 310 connects communication apparatus 312 and microcontroller 316, which in some aspects is attached to the casing, to sensor module 306. In some aspects, PCB 310 is used to provide instructions from communication apparatus 312 or power from battery 314 to sensor module 306. In certain aspects, PCB 310 allows communication apparatus 312 to transmit data either wirelessly or electronically to a processing apparatus which is external to portable device 300.

In some embodiments, an inlet, such as a sample well, allows a sample specimen of a subject to enter the sensor module. In one embodiment, a sensor module is positioned in the housing of a portable device to receive the sample specimen of the subject. In some aspects, the sensor module includes an array of sensors, e.g., biosensors, which may be independently capable of detecting the presence of one or more antibody compounds in the sample specimen of a subject. In some aspects, the array of biosensors is attached to a base, which may be molded plastic or ceramic. In some embodiments, the biosensors are selected to detect the presence of one or more antibody compounds in the sample specimen of the subject. In certain embodiments, sensor modules are replaceable, which allows the portable device to be rapidly adapted to recognizing the presence and identity of many different types of antibody compounds.

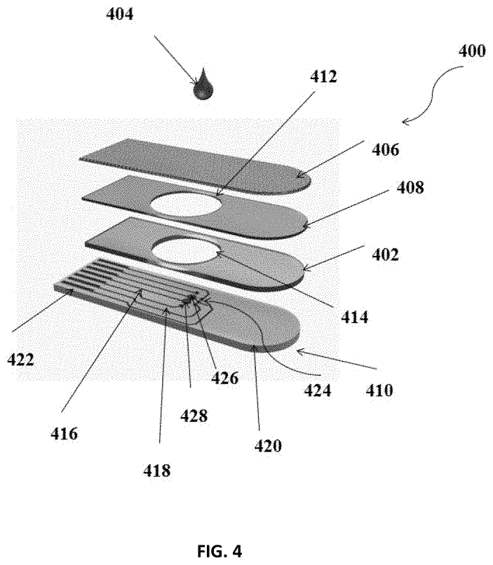

A micro fluidic, electrochemical biosensor array, as described in certain embodiments, is shown in FIG. 4. In some embodiments, a biosensor array 400, e.g., an electrochemical biosensor array, is a microfluidic system including amplification and detection reagents for electrochemical detection. In some embodiments, a device is configured to be insertable into an electrochemical reader for electronic readings. In some aspects, the detection of signals, such as electrical current, voltage, and other electric signals known in the art, is contemplated. In FIG. 4, electrochemical biosensor array 400 may include a first cellulosic layer 408 which includes one or more amplification agents in a sample deposition zone 412. In some embodiments, cellulosic layer (e.g., paper)-based test strips are used thereby reducing the sample volume and therefore cost of reagents. In some embodiments, the cellulosic layer is a patterned layer, e.g., a patterned paper layer. For example, the sample deposition zone 412 is a hydrophilic porous area in the cellulosic layer 408 defined by a fluid-impermeable material which permeates through the thickness of layer 408 and surrounds the sample deposition zone 412. In some embodiments, biosensor array 400 further includes a second cellulosic layer 410, with an electrode assembly 416 printed or attached onto layer 410. In one embodiment, electrode assembly 416 contains a one, two, three, four, five, six, seven, eight, nine, or ten electrodes. In some aspects, the electrode assembly includes one or more counter electrodes, one or more reference electrodes, and one or more working electrodes. In one embodiment, electrode assembly 416 contains a three electrodes system that includes counter electrode 428, reference electrode 426 and five working electrodes 424. In other embodiments, electrode assembly 416 contains 3, 4, or more electrodes, e.g., positive, negative and reference electrodes. The electrodes in assembly 416, e.g., screen-printed electrodes (SPE) 420, connect electrically to a set of contact pads 422 from the test zone 418. Optionally, a spacer layer 402 is disposed between layers 408 and 410. In certain embodiments, the spacer layer 402 is non-porous, e.g., plastic or glass. In certain embodiments, the spacer can be made from double-sided tape to join layers 408 and 410. In some aspects, spacer layer 402 includes an opening 414 to allow fluidic contact between portions of layers 408 and 410, e.g., the sample deposition zone 412 and the test zone 418. After a fluidic sample 404 is deposited in the sample deposition zone 412, an optional cover layer 406 may be placed on top of layer 408 to prevent fluid evaporation. In some aspects, sample deposition zone 412 is in fluidic communication with a test zone 418 on layer 410. In certain embodiments, one or more amplification agents are embedded in the test zone 418 and/or the sample deposition zone 412. In one embodiment, one or more amplification agents interact with the genetic material in the fluidic sample to provide copies of the genetic material using one or more methods described herein. In certain embodiments, one or more binding agents are embedded in the test zone 418 and/or the sample deposition zone 412 and are selected for binding the amplified genetic material to result in a change of the concentration of a signaling chemical. In some aspects, the test zone 418 is in fluidic communication with the electrode assembly 416. In some embodiments, a hydrophilic channel connects test zone 418 to electrode assembly 416 fluidically. In other embodiments, at least part of electrode assembly 416 is located in, printed in, or overlaps with test zone 418. In some embodiments, when the signaling chemical's concentration is changed or altered as a result of the binding between the binding agents and the amplified genetic material, such a change may be detected by the electrochemical reader or biosensor to generate an electronic readout. Other variations in the arrangement of the cellulosic layers, sample deposition zone and detection zone are contemplated and will be apparent to one of skill in the art.

In one embodiment, a simple electrochemical sensor identifies and detects one or more antibody compounds in a sample specimen, for example, glycoprotein P41, in the blood of a subject. In some aspects, a sample encounters an electrochemical sensor which may include a counter electrode, five working electrodes, and a reference electrode. The counter electrode is, for example, made of carbon paint, while the reference electrode is, for example, made of silver paint. In other aspects, counter and reference electrodes may be made of boron doped diamond, silver (Ag) or platinum (Pt). Electrodes which may be used in the sensors described herein include, but are not limited to, multiwalled carbon nanotubes (MWCNT)/Ag nanohybrids/Au, Ag nanoparticles/DNA/glassy carbon electrodes (GCE), nano-CuO/Ni/Pt, PtPdFe3O4 nanoparticles/GCE, Co3O4 nanoparticles/GCE, Cu2O/Ni/Au, AgMnO2-MWCNTs/GCE, immobilized with antibody with carbon nanostructure or graphene gold nanocomposites, etc. In some aspects, application of voltage to working electrodes, which includes carbon paint and multi-wall carbon nanotubes, leads to recognition of the presence and identity of one or more antibody compounds in the sample specimen of the subject.

Gold nanoparticles (AuNPs) are the most stable metal nanoparticles, due to their unique optical, electrical, and catalytic activity, as well as high biocompatibility properties and enhanced electron transfer rate. Therefore, they have shown wide applications in various electrochemical biosensors. Gold nanoparticles can be prepared by the chemical or electrochemical reduction of gold salt. Electrode deposition of AuNPs on the surface of carbon electrodes is appealing due to its direct, fast and easier preparation method.

Other materials which may be used in working electrodes include but are not limited to immobilized antibody gold nanoparticles, graphene, carbon nanotubes, reduced graphene, graphene oxide, carbon nanofibers, quantum dots, fullerene, carbon polymer nanocomposites, glassy carbon, carbon fiber nanocomposite, carbon black, etc. In the electrochemical biosensor illustrated in FIG. 4, the working electrodes are arrayed on a glass epoxy chip with an intervening insulating layer. Other supports for disposing the electrodes are generally known to those of skill in the art.

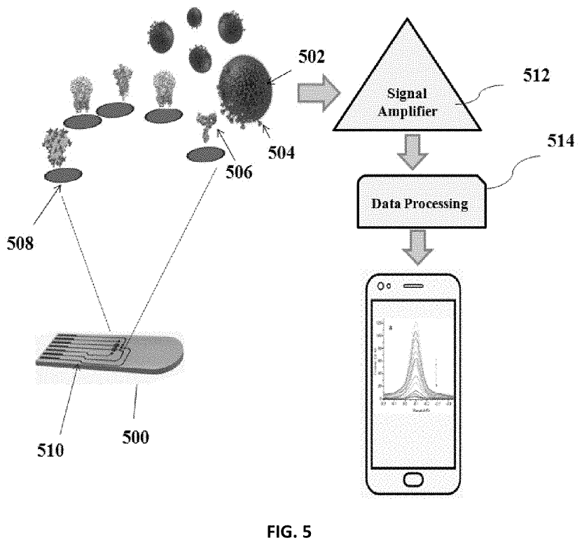

An exemplary sensor for use in a biosensor module as described herein is illustrated in FIG. 5. Sensor 500 includes one or more biological molecules 506 immobilized on support 508. Biological molecules or receptors 506 include, for example, enzymes, cells, protein receptors, antibodies, nucleic acids, etc. Exemplified in FIG. 5 are constituents 502 which are present in a specimen sample but do not bind to immobilized biological molecules 506 on support 508. Also shown in FIG. 5 is compound 504 which specifically binds to biological molecule 506. In some embodiments, upon binding of compound 504 to immobilized biological molecule 506, a signal is generated which is communicated from transducer 510 to communication apparatus (not shown) and then to processing apparatus 514, via signal amplifier 512, where the data is processed to confirm the presence and identity of the compound. In some aspects, signal amplifier 512 reduces instability and noise and may be purchased from commercial sources. Optionally, the signal may be directly communicated to the processing apparatus without the intermediacy of a signal amplifier.

In some embodiments, as seen in FIG. 4, a sensor, e.g., a biosensor, based on biological materials can be constructed by covalently attaching a biological molecule to the carbon nanostructure component of the working electrodes 424. In some embodiments, the biological molecule is an enzyme. In other embodiments, the biological molecule is an oxidase. In still other embodiments, the biological molecule is antibodies, spike protein or DNA. In one embodiment, the sensitivity of the antibody system, e.g., a glycoprotein p41 antibody system, is very high with the ability to detect some phenols at a sensitivity as low as 50 copies per ml. For example, glycoprotein p41 can be attached to an electrode by incubation with a binding agent for one to four hours, or in certain aspects for two hours, in buffer solution. The immobilized glycoprotein p41 antibody will be selectively interacting with glycoprotein p41 of HIV 1 in sample specimen solution as demonstrated by differential pulse voltammetry.

In some embodiments, DC voltage of 5V is applied directly to the microcontroller embedded in the sensor module which then transfers signals to the communication device and display device. Electrical signals may monitor on Mat lab in real-time fashion. Readings record in terms of electrical resistance. In some aspects, electrical resistance can be displayed into current and voltage gain if required by reconfiguring the program for the microcontroller.

An exemplary battery is a lithium ion battery, which are conventional and available from many commercial sources (e.g., Panasonic DMW-BCM14 battery). Many batteries are known in the art and may be used in the portable devices described herein.

In some embodiments, the processing apparatus will be a conventional general-purpose computer which includes a display device and a communication interface which allows reception and transmittal of information from other devices and systems via any communication interface. The processing module may detect and identify the antibody compounds in the sample specimen of the subject by processing the data received from the sensor module with results sent to the display device. Any general-purpose computer known in the art which has sufficient processing power to analyze data provided by the sensor module may be used in conjunction with the portable devices described herein.

In some embodiments, data from sensors in the sensor module is analyzed using pattern and recognition systems such as, for example, artificial neural networks, which include, for example, multi-layer perception, generalized regression neural network, fuzzy inference systems, etc., and statistical methods such as principal component analysis, partial least squares, multiples linear regression, etc. Artificial neural networks are data processing architectures that use interconnected nodes (i.e., neurons) to map complex input patterns with a complex output pattern. Importantly, neural networks can learn from using various input output training sets.

An exemplary artificial neural network can process data received from the sensor module. In general, the neural network can use three different layers of neurons. The first layer is input layer, which receives data from sensor module, the second layer is hidden layer while the third layer is output layer, which provides the result of the analysis. In some aspects, each neuron in hidden layer is connected to each neuron in input layer and each neuron in output layer. In the exemplified neural network, hidden layer processes data received from input layer and provides the result to output layer. Although only one hidden layer is described herein, any number of hidden layers may be used, with the number of neurons limited only by processing power and memory of the general-purpose computer or smart phone. In certain aspects, the inputs to the input neurons are inputs from the sensors in the sensor module. If, for example, seven sensors are in the sensor module, then the input layer will have seven neurons. In general, the number of output neurons corresponds to the number of compounds that the sensor module is trained to detect and identify. The number of hidden neurons may vary considerably. In some embodiments, the number of hidden neurons is between about 4 to about 10.

In some embodiments, the one or more compounds which are detected and identified using the portable devices are detected and identified directly. Pharmaceutical compounds which may be directly identified and detected include, but are not limited to, anti-gp41, anti-gp120, SC2A, IgG, anti-M2, BCN antibodies, monoclonal antibodies (MAbs), anti-CD4bs, ADCVI antibody, anti-HA, anti-FLAG, SARS-CoV-2-N antibody, SARS-CoV-2 nucleocapsid antibody, SARS-CoV-2-S antibody, SARS-CoV-2 N Ab (IgG), spike glycoprotein antibody, p21 antibody, ACE 2 antibody, gp150 antibody, CD147 antigen antibody, serine 2 antibody, MERS-CoV spike (S) protein, anti-HA antibody, anti-rhinovirus antibody, anti-HCV antibodies or HCoV-229E antigen protein. In certain embodiments, the one or more compounds which are detected and identified using the portable device are SARS-Cov-2 antibodies. For example, the one or more compounds which are detected and identified using the portable device are selected from the group consisting of SARS-CoV-2-nucleocapsid antibody, SARS-CoV-2-spike antibody, SARS-CoV-2 membrane antibody, and SARS-CoV-2 envelope antibody.

In some embodiments, the nanoparticle is chitosan and a polymer, polyvinyl alcohol nanoparticles or polyvinylpyrolidine nanoparticles, which may be made by methods well known in the art. In other embodiments, the polymer used with chitosan is tripolyphosphate, HPMC, HPC, PVP, ethyl cellulose, PEG, cellulose acetate phthalate and derivatives thereof, bioadhesive coatings such as, for example, poly(butadiene-maleic anhydride-co-L-DOPA) (PBMAD), etc.

The example mobile communication device shown in FIG. 5, can include one or more processors; a memory device including the biomarker processing module or engine, a diagnostic module, and an operating system (O/S); one or more activity sensors; a network and input/output (I/O) interface; and an output display. The example remote server computers can include one or more processors; a memory device including a biomarker processing module or engine, a diagnostic module, an operating system (O/S), and a database management system (DMBS); a network and input/output (I/O) interface; and an output display.

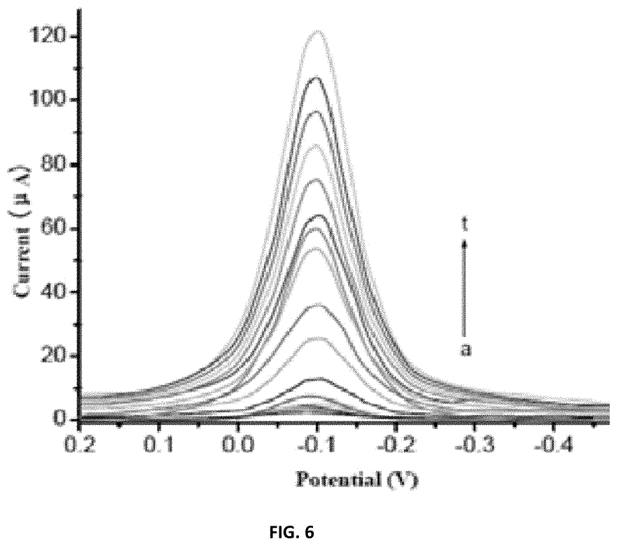

Those of skill in the art will appreciate that combinations of nanocomposite immobilized antibodies with antibody marker compounds at varying concentrations can be used to create a vast library of sequential viral detection. For example, glycoprotein p41, which includes the immobilized carbon nanostructure material, can be detected at different concentrations ranging from 50 copies per ml to 5×10⁶ copies per ml by differential pulse voltammetry and can be readily distinguished as illustrated in FIG. 6. FIG. 6 depicts an example user interface output by a diagnostic module, according to one example embodiment of the disclosure. In some embodiments, target biomolecules are also useful for diagnosis, monitoring virus diseases progression, and predicting disease recurrence. A virus target biomolecule refers to a substance or process that is indicative of the presence of same virus or not in the body.

In some embodiments, the devices and methods described herein are used to diagnose a subject as suffering from or having suffered from a viral infection. In certain embodiments, the devices and methods described herein are used to diagnose or identify a patient as having or had a coronavirus. For example, a subject may be diagnosed as suffering from or recovering from a SARS-CoV-2 infection. In one embodiment, a sample specimen from the subject is positive for SARS-CoV-2 antibodies. In certain embodiments, the sample specimen from the subject comprises one or more antibody compounds selected from the group consisting of SARS-CoV-2-nucleocapsid antibody, SARS-CoV-2-spike antibody, SARS-CoV-2 membrane antibody, and SARS-CoV-2 envelope antibody.

Many modifications and other embodiments of the example descriptions set forth herein to which these descriptions pertain will come to mind having the benefit of the teachings presented in the foregoing descriptions and the associated drawings. Thus, it will be appreciated that the disclosure may be embodied in many forms and should not be limited to the example embodiments described above. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A portable device for detecting and identifying one or more virus antibody compounds in a swab, saliva or blood specimen sample of a subject comprising:
a sensor module disposed in a housing, wherein the sensor module collects data for detecting and identifying one or more SARS-CoV-2 antibody compounds;
a biosensor array connected to the portable device, wherein the biosensor array comprises at least one biosensor comprising one or more carbon electrodes having SARS-CoV-2 antibodies immobilized with gold nanoparticles;
a communication apparatus connected to the sensor module, wherein the communication apparatus electrically transmits the data collected by the sensor module; and
a battery disposed to the communication apparatus and the sensor module.

2. The portable device of claim 1, further comprising a processing apparatus, wherein the processing apparatus processes the data collected by the sensor module and transmitted by the communication apparatus, thereby detecting and identifying the one or more antibody compounds, and wherein the processing apparatus is electronically or wirelessly connected to the communication apparatus.

3. The portable device of claim 2, wherein the processing apparatus transmits the identity of the one or more SARS-CoV-2 antibody compounds detected in the specimen sample to a display.

4. The device of claim 1, further comprising an amplifier, wherein the amplifier is connected to the sensor module, and wherein the amplifier amplifies data collected by the sensor module.

5. The device of claim 1, wherein the one or more SARS-CoV-2 antibody compounds are detected in real time.

6. (canceled)

7. (canceled)

8. The device of claim 1, wherein the one or more SARS-CoV-2 antibody compounds are selected from the group consisting of SARS-CoV-2-nucleocapsid antibody, SARS-CoV-2-spike antibody, SARS-CoV-2 membrane antibody, and SARS-CoV-2 envelope antibody.

9.-13. (canceled)

14. A method for detecting and identifying one or more SARS-CoV-2 antibody compounds in a sample specimen of a subject comprising:
transmitting the sample specimen of a subject collected on a biosensor array to a housing, wherein the biosensor array is connected to the housing, wherein the biosensor array comprises at least one biosensor comprising one or more carbon electrodes having SARS-CoV-2 antibodies immobilized with gold nanoparticles, and wherein the housing comprises a sensor module, a communication apparatus and a battery;
collecting data about the presence and identity of the one or more SARS-CoV-2 antibody compounds with the sensor module;
communicating the collected data via the communication apparatus to a processing apparatus; and
processing the communicated data to detect and identify the one or more SARS-CoV-2 antibody compounds in the sample specimen.

15. The method of claim 14, wherein the SARS-CoV-2 antibody compounds are selected from the group consisting of SARS-CoV-2-nucleocapsid antibody, SARS-CoV-2-spike antibody, SARS-CoV-2 membrane antibody, and SARS-CoV-2 envelope antibody.

16. The device of claim 1, wherein the at least one biosensor further comprises a binding agent and an amplification agent deposited on the one or more carbon electrodes.
